(19) United States
(12) Plant Patent Application Publication
(10) Pub. No.: US 2007/0079406 P1
(43) Pub. Date: Apr. 5, 2007

(54) Strawberry plant named 'Driscoll Ojai'

(76) Inventors: Amado Amorao, Camarillo, CA (US); Michael Ferguson, Moorpark, CA (US)

Correspondence Address: JONES DAY, 222 EAST 41ST ST, NEW YORK, NY, 10017, US

(21) Appl. No.: 11/244,121

(22) Filed: Oct. 4, 2005

(51) Int. Cl. A01H 5/00

(52) U.S. Cl. PLT/208

(57) ABSTRACT

This invention relates to a new and distinct variety of strawberry named ‘Driscoll Ojai’. The variety is similar to the varieties ‘Driscoll El Capitan’ and ‘Driscoll Venice’. The variety is distinguished from ‘Driscoll El Capitan’ and ‘Driscoll Venice,’ in particular, by its height, longer petiole length, longer fruiting truss length, open canopy density and moderate susceptibility to powdery mildew.

### LATIN NAME OF THE GENUS AND SPECIES OF THE PLANT CLAIMED

The variety is botanically identified as Fragaria×ananassa.

### VARIETY DENOMINATION

The strawberry variety denomination is ‘Driscoll Ojai’.

### BACKGROUND OF THE INVENTION

The new variety originated as a result of a controlled cross between the strawberry plants ‘Driscoll El Capitan’ (U.S. Plant Pat. No. 14,005) and ‘Driscoll Venice’ (U.S. Plant Pat. No. 14,062) in an ongoing breeding program, and was discovered as a seedling in Ventura County, California in 1999. The original seedling of the new cultivar was asexually propagated by stolons in a Nursery in Shasta County, California. Propagules were transplanted to a controlled breeding plot in Ventura County, California, where the variety was identified and selected for further evaluation. ‘Driscoll Ojai’ was subsequently asexually propagated and underwent further testing in Ventura County, California for four years. This propagation and testing has demonstrated that the combination of traits disclosed herein which characterize the new variety are fixed and retained true to type through successive generations of asexual reproduction.

### SUMMARY OF THE INVENTION

The present invention relates to a new and distinct variety of strawberry named ‘Driscoll Ojai’. The variety is botanically identified as Fragaria×ananassa. The new variety is distinguished from other varieties by a number of characteristics as set forth in Tables 1-4.

### Comparison to Similar Varieties

The varieties which we believe to be similar to ‘Driscoll Ojai’ from those known to us are ‘Driscoll El Capitan’ (U.S. Plant Pat. No. 14,005) and ‘Driscoll Venice’ (U.S. Plant Pat. No. 14,062). There are several characteristics of the new variety that are different from, or not possessed by ‘Driscoll El Capitan’ and ‘Driscoll Venice’. For example, the new variety is taller, has a longer petiole length, a longer fruiting truss length, an open canopy density and is moderately susceptible to powdery mildew.

‘Driscoll Ojai’ is distinguished from its parent, ‘Driscoll El Capitan’ and ‘Driscoll Venice’ as indicated in Tables 1-4.

### BRIEF DESCRIPTION OF THE DRAWINGS

The accompanying photographs show typical specimens of the new variety, including fruit, foliage and flowers, in color as nearly true as it is reasonably possible to make in color illustrations of these characteristics.

FIG. 1 shows the whole the plant.

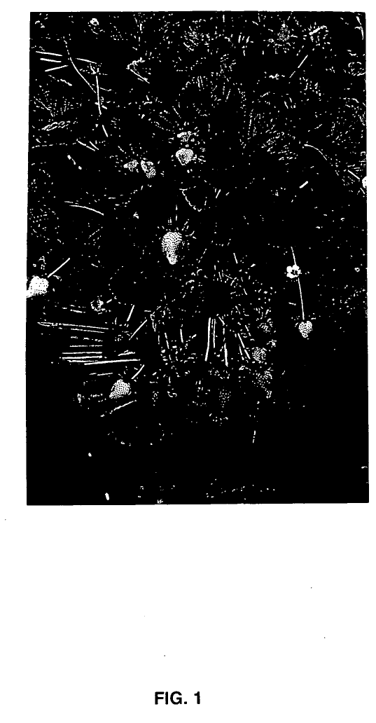

FIG. 2 shows the upper side of the leaves of the plant.

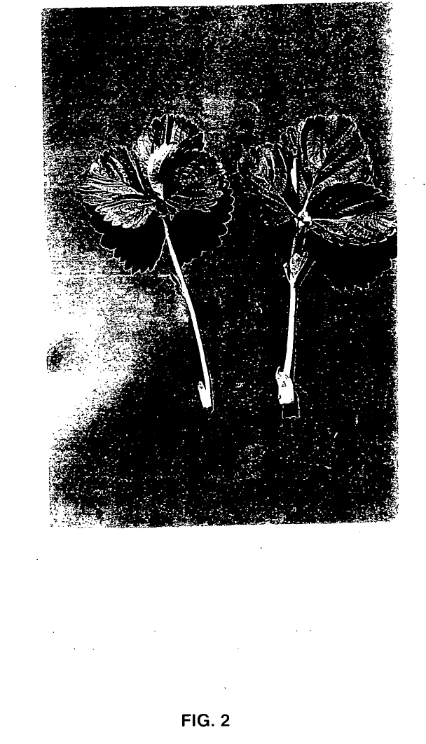

FIG. 3 shows the under side and upper side of the flowers.

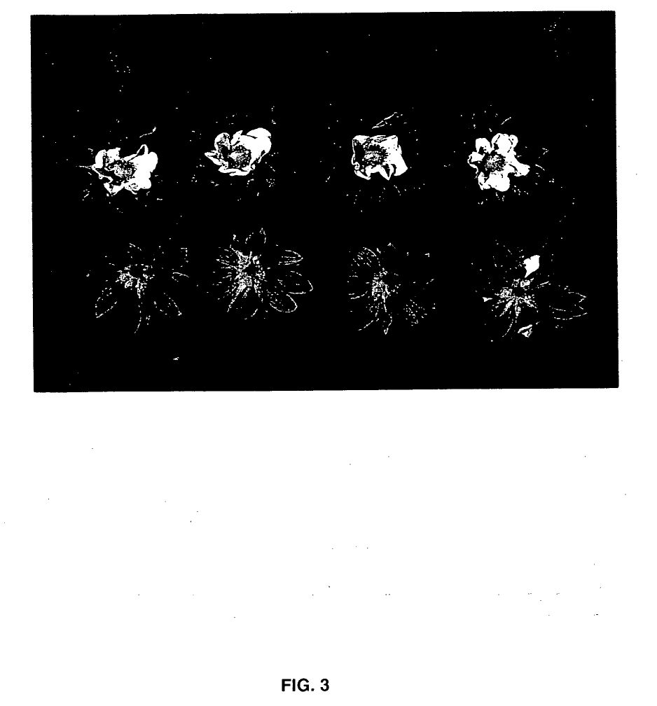

FIG. 4 shows a close-up of the fruit.

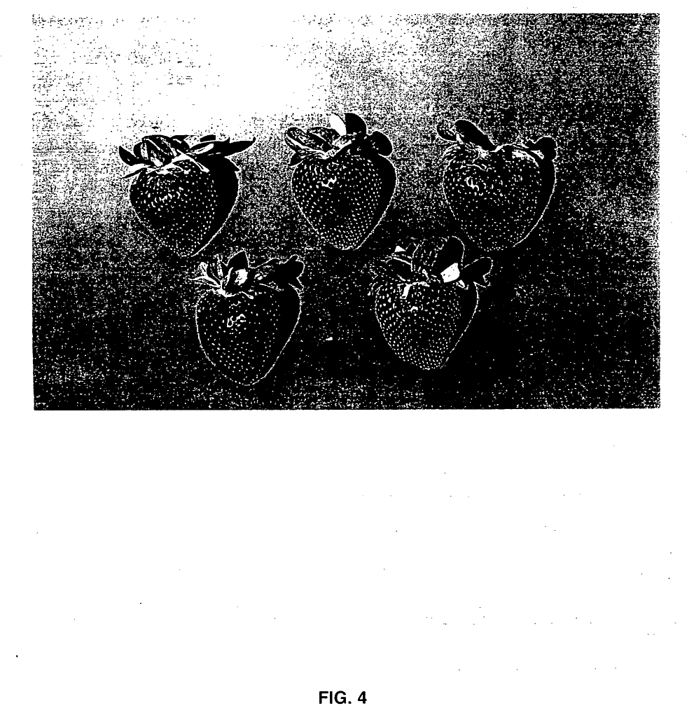

FIG. 5 shows the fruit in longitudinal cross-section.

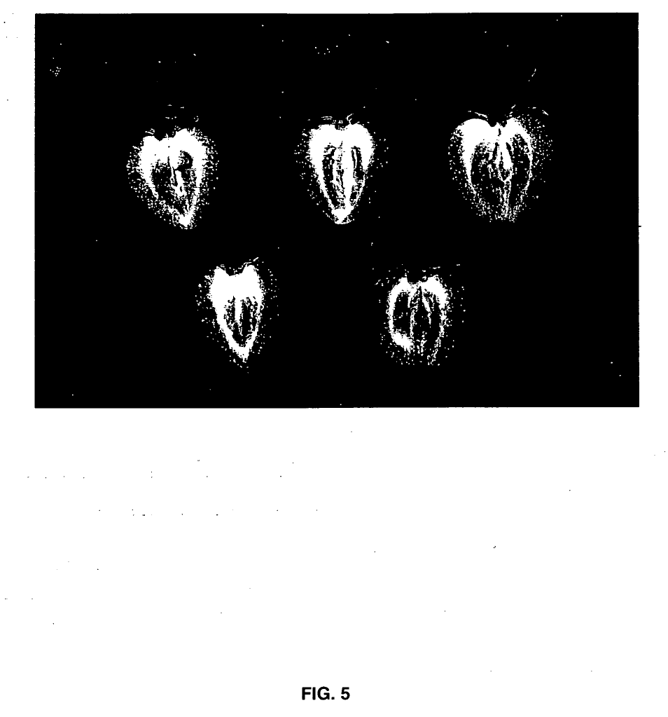

### DESCRIPTION OF THE NEW VARIETY

The following detailed description of the new variety is based upon observations taken of plants harvested from a nursery in Bonanza, Oreg. in September and October, 2004 and held in cold storage until planting in Ventura County, California in October, 2004. Plants were grown in raised beds of soil under conditions typical of commercial strawberry production in Ventura County, California. Fruits were harvested twice weekly from January, 2005 to May, 2005 for yield determination. Observations of ‘Driscoll Ojai’, ‘Driscoll El Capitan’, and ‘Driscoll Venice’ were made in side by side comparison in April, 2005. Color designations, color descriptions, and other phenotypical descriptions may deviate from the stated values and descriptions depending upon variation in environmental, seasonal, climatic and cultural conditions. Colors are described and the most similar color designations are provided from The Royal Horticultural Society (RHS) Colour Chart.

### Propagation

The new variety is principally propagated by way of stolons. Although propagation by stolons is presently preferred, other known methods of propagating strawberry plants may be employed.

### Characteristics of the New Variety

Information on the new variety is presented in Tables 1, 2, 3 and 4. In the tables, the flowers described are secondary flowers except where indicated. The fruit described is the secondary fruit on seven month old plants. Fruit and flower measurements are an average of both primary and secondary fruit and flowers.

Table 1 provides information on the plant and fruit characteristics of the new variety ‘Driscoll Ojai’ compared with characteristics of ‘Driscoll El Capitan’ and ‘Driscoll Venice’. Table 2 provides additional information of the plant and fruit characteristics of the new variety ‘Driscoll Ojai’ compared with characteristics of the varieties ‘Driscoll El Capitan’ and ‘Driscoll Venice’. Table 3 provides reactions of the new variety to stresses, pests and diseases compared with reactions of the varieties ‘Driscoll El Capitan’ and ‘Driscoll Venice’. Table 4 provides isozyme characteristics of the new variety as compared to that of the varieties ‘Driscoll El Capitan’ and ‘Driscoll Venice’.

TABLE 1
QUANTITATIVE COMPARISON OF ‘DRISCOLL OJAI’,
‘DRISCOLL EL CAPITAN’ AND ‘DRISCOLL VENICE’

| | ‘Driscoll | ‘Driscoll El | ‘Driscoll |
|---|---|---|---|
| | Ojai’ | Capitan’ | Venice’ |
| Plant Characteristics | | | |
| Height of Plant (cm) | 22.6 | 20.8 | 18.5 |
| Spread of Plant (cm) | 32.1 | 31.0 | 31.5 |
| Number of Crowns | 4.1 | 4.0 | 3.2 |
| Leaf Characteristics | | | |
| Terminal Leaflet Length | 7.4 | 6.7 | 6.3 |
| (cm) | | | |
| Terminal Leaflet Width | 7.0 | 6.0 | 6.5 |
| (cm) | | | |
| Terminal Leaflet | 1.06 | 1.12 | 0.96 |
| Length/Width Ratio | | | |
| # Teeth/Terminal Leaflet | 22.0 | 20.0 | 23.7 |
| Color of upper side | 137A | 147A | 147A |
| | medium | dark green | dark |
| | green | | green |
| Color of under side | 147D | 147C | 139C |
| | light to | light to | light to |
| | medium | medium | medium |
| | green | green | green |
| Petiole Length (cm) | 15.5 | 13.5 | 12.2 |
| Petiole Diameter (mm) | 3.0 | 3.1 | 3.5 |
| Petiole Color | 145C | 149A | 149A |
| | yellow green | yellow green | yellow green |
| Petiolule Length (mm) | 7.6 | 8.1 | 5.9 |
| Petiolule Diameter (mm) | 1.8 | 1.7 | 1.8 |
| Petiolule Color | 145B | 149A | 149A |
| | yellow green | yellow green | yellow green |
| Bract Frequency | 42% | 0% | 8% |
| | typically | | typically |
| | paired | | single |
| Stipule Length (cm) | 3.8 | 3.9 | 3.7 |
| Stipule Width (cm) | 0.94 | 0.93 | 0.95 |
| Stolon Characteristics | | | |
| Anthocyanin color | 61C | 58A | |
| | purple red | purple red | |
| Diameter at bract (mm) | 3.16 | 2.51 | |
| Avg. # of Daughter plants | | | |
| (2003 Nursery) | | | |
| Flower Characteristics | | | |
| Petal Length (cm) | 1.28 | 1.31 | 1.40 |
| Petal Width (cm) | 1.23 | 1.28 | 1.32 |
| Petal Length/Width Ratio | 1.04 | 1.02 | 1.06 |
| Flower Diameter (cm) | 2.35 | 2.37 | 2.6 |
| Calyx Diameter (cm) | 4.3 | 4.24 | 4.94 |
| Sepal Length (mm) | 19.6 | 18.6 | 20.9 |
| Sepal Width (cm) | 9.49 | 8.33 | 9.67 |
| Petal Color | 155B | 155C | 155C |
| | white | white | white |
| Receptical Color | 1A | 7A | 7A |
| | yellow | yellow | yellow |
| Anther Color | 12A | 13A | 13A |
| | yellow | yellow | yellow |
| Fruiting Truss Length (cm) | 27.9 | 24.8 | 18.2 |
| Fruit Characteristics | | | |
| Fruit Length (cm) | 4.6 | 4.3 | 4.5 |
| Fruit Width (cm) | 3.7 | 3.3 | 4.2 |
| Fruit Length/Width Ratio | 1.27 | 1.32 | 1.06 |
| Average Berry Weight (g) | 26.8 | 26.3 | 30.6 |
| External Color | 46A | 53A | 45A |
| | dark red | dark red | medium red |
| Internal Color | 42B | 43A | 34A and 155C |
| | medium red | red | orange red and |
| | | | white |
| Achene Coloration | 59B to 9A | 60A to 151A | 60A to 15B |
| | dark red to | dark red to | dark red to |
| | yellow | yellow green | yellow |
| 2003 Marketable Yield | 712 | 661 | 592 |
| (g/plant) | | | |

TABLE 2
QUALITATIVE COMPARISON OF ‘DRISCOLL OJAI’,
‘DRISCOLL EL CAPITAN’ AND ‘DRISCOLL VENICE’

| | Driscoll | ‘Driscoll | ‘Driscoll |
|---|---|---|---|
| | ‘Ojai’ | El Capitan’ | Venice’ |
| Plant | | | |
| Habit | globose | globose | globose |
| Canopy Density | open | medium | medium |
| | | dense | dense |
| Vigor | strong | medium | medium |
| Leaf | | | |
| Shape in cross section | concave | slightly | slightly |
| | | concave | concave |
| Interveinal blistering | strong | strong | strong |
| Glossiness | medium | strong | strong |
| Number of leaflets | three | three | three |
| | only | only | only |
| Terminal leaflet margin profile | flat | flat | flat |
| Terminal leaflet shape of base | rounded | rounded | rounded |
| Terminal leaflet shape of teeth | obtuse | obtuse | obtuse |
| Stipule pubescence | medium | sparse to | medium |
| | | medium | |
| Petiole pubescence | dense | dense | dense |
| Petiole pose of hairs | outwards | outward to | outward to |
| | | downward | downward |
| Stolon | | | |
| Anthocyanin coloration | weak | medium to | medium |
| | | strong | |
| Thickness | medium | thin | medium |
| Pubescence | weak | sparse to | medium to |
| | | medium | dense |
| Inflorescence | | | |
| Position relative to foliage | above | above | level to |
| | | | above |
| Diameter of calyx relative to | larger | larger | larger |
| corolla on secondary flowers | | | |
| Diameter of inner calyx relative to | larger | smaller to | smaller |
| outer on secondary flowers | | same size | |
| Spacing of petals | touching | over- | touching |
| | | lapping | to over- |
| | | | lapping |
| Fruiting Truss | | | |
| Attitude at first picking | prostrate | prostrate | prostrate |
| Fruit | | | |
| Predominant shape | almost | cordate | cordate |
| | cylindrical | | |
| Difference in shapes between | moderate | marked | slight |
| primary and secondary fruits | | | |
| Band without achenes | narrow | very | very |
| | | narrow | narrow |
| Unevenness of surface | weak to | weak to | weak |
| | medium | medium | |
| Evenness of color | even | slightly | slightly |
| | | uneven | uneven |
| | | to even | to even |
| Glossiness | strong | strong | medium to |
| | | | strong |
| Insertion of achenes | level with | below to | level with |
| | surface | level with | surface |
| | | the surface | |
| Insertion of calyx | level | in a basin | level to |
| | | to level | set above |
| Pose of the calyx segments | spreading | reflexed | reflexed |
| | to reflexed | | |
| Size of calyx in relation to fruit on | same size | larger | larger |
| secondary fruit | to larger | | |
| Adherence of calyx | medium to | strong | strong |
| | strong | | |
| Firmness of flesh | firm | firm | medium |
| Evenness of flesh color | slightly | even | uneven |
| | uneven | | |
| Distribution of flesh color | marginal | marginal | only |
| | and | and | marginal |
| | central | central | |
| Hollow center size | medium | large | small to |
| | | | medium |
| Sweetness | strong | strong | strong |
| Texture when tasted | fine to | fine | medium |
| | medium | | |
| Acidity | medium | medium | weak |
| Time of First Flowering After | mid- | mid- | late- |
| Planting in 2004 | December | December | December |
| Harvest Interval in 2005 | late | late | late |
| | January | January | January |
| | to | to | to |
| | late-May | late-May | late-May |
| Type of Bearing | partially | partially | partially |
| | ever- | ever- | ever- |
| | bearing | bearing | bearing |

### Reaction to Stress, Pests, and Disease

TABLE 3
REACTIONS TO STRESS PESTS AND DISEASES
FOR ‘DRISCOLL OJAI’, ‘DRISCOLL EL
CAPITAN’ AND ‘DRISCOLL VENICE’

| | | ‘Driscoll | ‘Driscoll |
|---|---|---|---|
| | ‘Driscoll Ojai’ | El Capitan’ | Venice’ |
| Reaction to Pests | | | |
| Tetranychus urticae | moderately | moderately | moderately |
| | resistant | resistant | resistant |
| Lygus hesperus | susceptible | susceptible | susceptible |
| Reaction To Diseases | | | |
| Botrytis fruit rot | moderately | moderately | moderately |
| | susceptible | susceptible | susceptible |
| Powdery mildew | moderately | moderately | moderately |
| | susceptible | resistant | resistant |
| Verticillium wilt | susceptible | susceptible | susceptible |
| Strawberry Mottle Virus | moderately | moderately | moderately |
| | resistant | resistant | resistant |
| Xanthomonas fragariae | moderately | moderately | moderately |
| | susceptible | susceptible | susceptible |

### Isozyme Analysis

In addition to the morphological description above, the new cultivar ‘Driscoll Ojai’ has been analyzed to obtain an indication of its genetic makeup to provide further means for identifying the new variety and distinguishing it from other somewhat similar and/or related strawberry varieties. Specifically, leaf samples of ‘Driscoll Ojai’, ‘Driscoll El Capitan’ and ‘Driscoll Venice’ were analyzed by electrophoresis for isozyme patterns of the enzymes phosphoglucoisomerase (“PGI”), leucine aminopeptidase (“LAP”) and phosphoglucomutase (“PGM”). See J. Amer. Soc. Hort. Sci. 106:684-687. Isozyme characterization of the three varieties is presented in Table 4, with the letters representing the banding patterns for each enzyme as designated in the above-identified article.

TABLE 4
ISOZYME ANALYSIS FOR ‘DRISCOLL OJAI’, ‘DRISCOLL EL
CAPITAN’ AND ‘DRISCOLL VENICE’

| | ‘Driscoll | ‘Driscoll El | ‘Driscoll |
|---|---|---|---|
| Locus | Ojai’ | Capitan’ | Venice’ |
| PGI | A1 | A2 | A4 |
| LAP | B3 | B3 | B3 |
| PGM | C2 | C4 | C2 |

What is claimed:

1. A new and distinct variety of strawberry plant, substantially as shown and described.
